(19) United States
(12) Patent Application Publication
(10) Pub. No.: US 2023/0358645 A1
(43) Pub. Date: Nov. 9, 2023

(54) APPARATUS AND METHOD FOR DETERMINING OPERATING PARAMETERS FOR DEGASSING AND/OR DEODORIZING PLASTIC PARTICLES

(71) Applicant: Coperion GmbH, Stuttgart (DE)

(72) Inventors: Klaus-Peter LANG, Bad Waldsee (DE); Christian DIKREUTER, Immenstaad (DE)

(73) Assignee: Coperion GmbH, Stuttgart (DE)

(21) Appl. No.: 18/311,142

(22) Filed: May 2, 2023

(30) Foreign Application Priority Data
May 4, 2022 (DE) 10 2022 204 421.5

(51) Int. Cl. G01N 1/22; G01N 33/44

(52) U.S. Cl. CPC G01N 1/22; G01N 33/445; G01N 2001/2229

(57) ABSTRACT

An apparatus for determining operating parameters for degassing and/or deodorizing plastic particles comprises at least one test chamber for degassing and/or deodorizing a test quantity of plastic particles, wherein each test chamber has a filling opening for filling the test quantity, a withdrawal opening for withdrawing bulk material, a vent opening for venting the test chamber, wherein a pressurized gas line for feeding pressurized gas and a heating element and/or a steam line for feeding steam and a steam generator are connected to a feed opening of the test chamber.

### CROSS-REFERENCES TO RELATED APPLICATIONS

This application claims the priority of German Patent Application, Serial No. DE 10 2022 204 421.5, filed May 4, 2022, the content of which is incorporated herein by reference in its entirety as if fully set forth herein.

### FIELD OF THE INVENTION

The invention relates to an apparatus and a method for determining operating parameters for degassing and/or deodorizing plastic particles.

### BACKGROUND OF THE INVENTION

It is known that plastic particles can contain volatile components, in particular monomers, moisture, gases, in particular air, solvents, odors and flavors such as limonene and/or volatile organic compounds (VOC). Plastic particles are categorized as, for example, plastic granulate, plastic flakes and plastic powder, depending on the particle size and/or particle shape. For the sake of simplicity, the plastic particles will be referred to as bulk material in the following. In order to remove the volatile components, the bulk material can be degassed and/or deodorized, for example, in a mixing device according to DE 10 2012 206 017 A1. When the bulk material is being degassed and/or deodorized, it is flushed with a gas, in particular air, in order to remove the volatile components. The operating parameters required for this, such as temperature, pressure, quantity of air, humidity and degassing duration, are typically determined by experience and/or experiment and depend not least on the bulk material to be degassed. The determination of suitable operating parameters is complex. Changing quality grades of the bulk material to be processed increase the effort required to determine the operating parameters for degassing and/or deodorization enormously. In particular, bulk material made of plastic recyclate can have particularly frequently changing quality grades. Degassing and/or deodorizing plastic bulk material is time-consuming, energy-intensive and thus cost-intensive.

### SUMMARY OF THE INVENTION

It is an object of the present invention to simplify the degassing and/or deodorization of bulk material and, in particular, to reduce the effort required to determine the corresponding operating parameters.

This object is achieved according to the invention by an apparatus for determining operating parameters for degassing and/or deodorizing plastic particles, wherein the apparatus comprises at least one test chamber for degassing and/or deodorizing a test quantity of plastic particles, wherein each test chamber has a filling opening for filling in the test quantity, a withdrawal opening for withdrawing a sample quantity of the plastic particles, a vent opening for venting the test chamber, wherein connected to a feed opening of the test chamber are a pressurized gas line for feeding pressurized gas and a heating element and/or, a steam line for feeding steam and a steam generator and by a method for determining operating parameters for degassing and/or deodorizing plastic particles comprising the method steps of providing an apparatus according to the invention, filling a test quantity of plastic particles into the test chamber, degassing and/or deodorizing the test quantity in the test chamber by feeding temperature-controlled pressurized gas and/or steam via a feed opening of the test chamber, removing a sample quantity of the degassed and/or deodorized plastic particles from the test chamber, determining material properties of the sample quantity, determining operating parameters for degassing and/or deodorizing on the basis of the determined material properties.

According to the invention, it has been recognized that relevant operating parameters for degassing and/or deodorizing plastic particles can be determined by means of a suitable test quantity in an apparatus provided for this purpose. The apparatus is in particular a test rig, which is in particular compact and in particular transportable, i.e. mobile. Mobile means in particular that the test rig itself can be movable, in particular drivable. For this purpose, the test rig can have at least one and in particular several castors and/or wheels. The castors and/or wheels are arranged in particular on an underside of a rack of the test rig and are designed in particular to be height-adjustable. The castors and/or wheels can be articulated to the test rig so as to be rotatable about a vertical axis and/or lockable. To improve the handling of the mobile test rig, the test rig may have one or more handles. The test rig can be conveniently pushed and/or pulled. The at least one handle can be designed to be removable.

The determination of the operating parameters is not complex and is possible in particular flexibly and/or as required.

The apparatus can be used to prepare and analyze the test quantity of plastic particles, in particular in advance, in order to determine the operating parameters, i.e. degassing parameters for a preparation process. These operating parameters can be set at a degassing silo of a compounding and/or recycling facility. Additionally or alternatively, the apparatus can be used to dimension new facilities for certain plastic particles, in particular optimally, already during their planning phase. A suitable test quantity is between 1 l and 100 l, in particular between 5 l and 50 l, in particular between 5 l and 20 l, in particular between 8 l and 15 l and in particular 10 l, i.e. 0.01 m³. It has been found that this volume of the test quantity is sufficient to realistically represent, i.e. simulate, the degassing and/or deodorization process. The test quantity is therefore sufficiently large so that scaling effects compared to a real compounding and/or recycling facility are negligible.

The bulk material comprises individual plastic particles, in particular granules with an average particle size between 0.5 mm and 6.0 mm, in particular between 0.7 mm and 4.0 mm Plastic particles can also be powder particles with an average particle size between 1 μm and 2,000 μm, in particular between 10 μm and 2,000 μm, in particular between 50 μm and 2,000 μm, in particular between 50 μm and 200 μm, in particular between 70 μm and 200 μm and in particular between 70 μm and 150 μm, and/or flake-like particles, so-called flakes, with an average edge length between 1.0 mm and 30.0 mm, in particular between 2.0 mm and 25.0 mm and in particular between 5.0 mm and 20.0 mm.

Polyolefins, in particular polyethylene terephthalate (PET) and/or polystyrene (PS), serve as the plastic material.

It has been found that in a test chamber of the apparatus, operating conditions for degassing and/or deodorizing can be set that correspond to the real operating conditions in a silo of a compounding and/or recycling facility.

In the test chamber, the operating parameters corresponding to a large degassing silo can thus be simulated, although the degassing silo has a volume that is significantly larger than the volume of the test chamber. In particular, the volume of the test chamber is at most 0.1 times, in particular at most 0.01 times, in particular at most 0.005 times, in particular at most 0.001 times and in particular at most 0.0001 times the volume of a degassing silo.

The apparatus has at least one test chamber. If several test chambers are provided, it is in particular possible to simulate different operating conditions in parallel. This can reduce the time required to determine suitable operating conditions. The test chambers are in particular designed to be comparable and in particular identical to each other. In particular, the test chambers each have an identical size and, in particular, allow test quantities of identical volume to be accommodated. The test chambers can also have volumes of different sizes. Functionally, the test chambers are designed identically.

The real operating parameters can be determined quickly and easily using a comparatively small test quantity. The test chamber has a filling opening, in particular on its upper front side, to fill in the test quantity.

The test quantity that is arranged in the test chamber can be impinged on with a gas for degassing. Air, in particular pressurized air, serves as the gas. However, other gases can also be used, in particular nitrogen. It is advantageous if the pressurized gas is inert.

The test chamber has a feed opening for feeding pressurized gas, in particular pressurized air, from a pressurized gas line into the test chamber. The apparatus enables feeding of heated gas into the test chamber. A heating element is used in particular for heating the gas and is connected to the pressurized gas line. The heating element is in particular designed as an electric heater, in particular as an air heater. The air heater can also be used for heating inert gases, in particular nitrogen. With regard to the pressurized gas line, the feed opening constitutes a pressurized gas connection to the test chamber.

The apparatus makes it possible to impinge on the test quantity in the test chamber with hot pressurized gas, in particular with hot air, having a temperature of ambient temperature up to 150° C. In particular, the apparatus allows temperatures of at least 50° C., in particular of at least 70° C., in particular of at least 90° C., in particular of at least 100° C., in particular of at least 120° C. to be impinged on.

In order to implement different moisture contents in the test chamber, steam, in particular water vapor, can be fed into the test chamber by means of a steam line. A steam generator that can evaporate water is connected to the steam line. The steam line is connected to the feed opening of the test chamber. In relation to the steam line, the feed opening constitutes a steam connection.

In particular, the steam line and the pressurized gas line open into the same feed opening. In particular, the two lines open into each other upstream of the feed opening, so that only one line is connected to the feed opening. This line section that is connected to the feed opening, which is in particular a part of the steam line or the pressurized gas line, is also referred to as gassing line.

One finding of the invention is based on the fact that heated pressurized gas and/or water vapor can be fed into the test chamber through the feed opening in order to determine the operating parameters.

The test chamber has a withdrawal opening, in particular laterally, for withdrawing a sample quantity of the plastic particles from the test chamber. In particular, the sample quantity forms a partial quantity of the test quantity. The sample quantity has a volume of 20 ml to 200 ml, for example. Withdrawing the sample quantity serves to determine the degassing and/or deodorization progress and thus to verify the previously applied operating parameters. The operating parameters include in particular temperature, pressure, quantity of air, moisture content and process duration, i.e. gassing duration. In particular, multiple test series are carried out with the apparatus in order to determine the operating parameters. The execution of a test can be performed in an uncomplicated manner

It has been found that the apparatus according to the invention can be of small design, i.e. compact. In particular, the test chamber has a maximum volume of at most 100 l, in particular at most 50 l, in particular at most 20 l and in particular 10 l. In particular, the apparatus is designed to be mobile, i.e. transportable. For example, the apparatus can be transported in a van. In particular, the apparatus is designed in such a manner that protruding attachments such as lines, pipes, cables and/or housing parts can be easily removed for transporting the apparatus. In particular, it is possible to transport the apparatus directly to the compounding and/or recycling facility and to immediately determine the operating parameters for the respective bulk material to be conveyed. In particular, the apparatus can be designed in such a manner that it can be lifted and moved using common lifting and/or conveying means, such as a forklift truck or lift truck. For this purpose, the apparatus can be transported by means of a, in particular standardized, transport pallet, a so-called EPAL Euro pallet.

In the transport state, a base surface of the apparatus is in particular rectangular or can be arranged in a rectangle which has a maximum length of 1.8 m, in particular of at most 1.6 m and in particular of at most 1.5 m. The rectangle surrounding the base surface of the apparatus has a maximum width of at most 1.5 m, in particular of at most 1.3 m and in particular of at most 1.2 m. A maximum height of the apparatus in the transport state is at most 1.6 m, in particular at most 1.5 m and in particular at most 1.4 m.

In particular, the mass of the apparatus is less than the maximum load capacity of a Euro pallet. In particular, the maximum mass of the apparatus is at most 2.0 t, in particular at most 1.5 t and in particular at most 1.0 t.

In particular, the test chamber has a main vent. A vent opening is arranged at the test chamber, in particular at its upper end face, to which in particular a vent line can be connected. The vent line is designed in particular as a chimney pipe. The vent line is in particular attached to the vent opening in a removable manner The removable vent line enables a particularly compact design of the test chamber. Hot and in particular harmful vapors, in particular vapors containing PVC components, can be reliably removed from the test chamber via the main vent, in particular above an operator's working level. With the vent line, the test chamber can be connected to a central venting device.

In particular, a central power supply is provided for the power supply of the apparatus. This can be a corresponding electrical connection which enables a connection to a central voltage supply, in particular a line network.

Alternatively, a corresponding device, in particular a generator and/or a rechargeable battery, can be provided at the apparatus, which provides the required voltage supply, in particular for a mobile and, in particular, a self-sufficient use of the facility.

An apparatus comprising a gas flow regulator that is connected to the pressurized gas line enables a flexible, in particular need-based, adjustment, in particular regulation, of the supplied gas quantity and/or the gas pressure in the test chamber. A gas flow regulator enables in particular the regulation of a gas flow, in particular an air flow, which serves in particular to heat or keep warm the housing of the test chamber. The gas flow and/or gas pressure regulation can be performed manually and/or automatically, in particular fully automatically. Along the gas flow direction, the gas flow regulator is arranged in particular downstream with respect to the heating element. The arrangement of the gas flow regulator and heating element along the gas flow direction can also be reversed.

An apparatus comprising a pressurized gas source that is connectable to the pressurized gas line ensures a reliable pressurized gas supply. It is particularly advantageous if the pressurized gas line can be connected to a pressurized gas source in the form of a central pressurized air network. Such a pressurized air network provides pressurized gas, in particular pressurized air, in a range of 4 bar to 8 bar and is often present in any event in commercial facilities, for example. A separate pressurized gas source is dispensable. Existing resources can be used directly.

Alternatively or additionally, it is possible to use a compressor which is connected to the pressurized gas line as the pressurized gas source. A compressor as a pressurized gas source increases the flexibility of use of the apparatus and enables self-sufficient operation of the apparatus.

A vent opening enables safe operation of the steam generator.

It is advantageous if the steam generator has a vent opening to which a vent line, in particular in the form of a vent pipe, is connected. The vent pipe is in particular oriented vertically and has a length in particular such that steam is discharged from the steam generator in a height section which is arranged above a head height of an operator, in particular at a height of at least 2.5 m above the floor. The vent line can be dismantled for transport.

A water softening unit that is connected to the steam generator enables the use of drinking water containing lime, in particular an existing drinking water pipe network, for steam generation.

A steam volume regulator that is connected to the steam line enables the variably determinable addition of moisture into the test chamber. The steam volume regulator can be operated manually or automatically, in particular fully automatically. The steam volume regulator can be used to selectively regulate a steam flow.

A water source that is connectable to the steam generator ensures the direct addition of moisture into the test chamber. It is particularly advantageous if an already existing pipe network can be used for the water supply. In addition or alternatively, the apparatus can have a water tank and, in particular, a pump, in order to enable self-sufficient operation of the apparatus.

A vent line that is connectable to the vent opening for connecting the test chamber to a central venting system ensures a reliable and safe discharge of gases from the test chamber. By means of the at least one vent line, the test chamber can be reliably connected to a venting system, in particular an existing one.

A withdrawal unit connected to the one withdrawal opening, which withdrawal unit comprises in particular a withdrawal pipe, in particular a downpipe, which can be shut off by means of a shut-off element, enables a quantity of bulk material to be withdrawn reliably and, in particular, with repeat accuracy in order to verify the operating parameters previously applied in the test chamber. A quantity of bulk material that has been withdrawn by means of the withdrawal unit can be examined and analyzed in more detail. It is particularly advantageous if the withdrawal unit has a withdrawal pipe that can be shut off, in particular in the form of a downpipe. The design of the withdrawal unit is reliable, robust and uncomplicated. In particular, the downpipe is inclined with respect to the vertical by an angle of inclination which is in particular at most 30°, in particular at most 20°, in particular at most 15°, in particular at most 10° and in particular at least 5°.

A control unit which is designed to control operating parameters, in particular temperature, pressure and/or amount of pressurized gas supplied, water content and/or degassing time for degassing and/or deodorizing the test amount in the test chamber simplifies the execution of test series with different operating parameters. In particular, the control unit ensures the execution of a degassing cycle with operating parameters that can be set variably, i.e. temperature, pressure and/or quantity of the supplied pressurized gas, water content, i.e. moisture content, and degassing duration. The control unit has a signal connection, in particular a bidirectional signal connection, in particular with the heating element, the gas flow regulator, the steam generator, the steam volume regulator and/or a temperature control unit.

A temperature control unit for controlling the temperature of an outer wall of the test chamber, wherein the temperature control unit comprises a double-walled housing surrounding the test chamber or a separate heater ensures constant temperatures in the test chamber, in particular on an inner side of the housing of the test chamber. The temperature control unit serves to control the temperature of an outer wall of the housing. The temperature control unit is arranged outside and in particular directly on the outer wall of the housing. It has been found that this can reduce and in particular avoid temperature influences on the test quantity within the test chamber as a result of wall influences. In particular, the test quantity in the test chamber has a homogeneous temperature distribution. In particular, the temperature control unit can minimize the risk of a temperature gradient towards the outer wall of the test chamber. A temperature difference between the test quantity and the outer wall of the test chamber is minimized and is at most 30 K, in particular at most 20 K, in particular at most 10 K and in particular at most 5 K.

The design of the temperature control unit with a double-walled housing in the form of an outer pipe surrounding the test chamber is particularly advantageous. The outer pipe can be filled with a heated fluid and in particular with heated gas, in particular with heated air. In particular, the pressurized gas line for feeding heated pressurized gas is connected to the double-walled housing. With this design of the temperature control unit, it can be ensured in particular that the outer pipe of the test chamber has the same temperature as the bulk material that is fed into the test chamber. A further vent opening with a further vent line can be connected to the double-walled housing. The further vent opening with the further vent line constitute a secondary vent. The further vent line can in particular be dismantled for the transport state. The steam emitted via the secondary vent is in particular harmless to health. In particular, hot air and/or pure water vapor are discharged via the secondary vent.

Instead of the double-walled design of the housing, the temperature control unit can have a separate heater to heat the outer wall of the housing of the test chamber. This separate heater, in particular in the form of a heating blanket, can be attached directly to the outer wall of the housing of the test chamber.

The separate heater, which is operated in particular with electric current, can also be used in particular to preheat the test chamber, in particular to improve the test results when the degassing and/or deodorization is carried out for the first time, i.e. in a so-called first test.

An apparatus comprising a rack supporting the apparatus, to which rack in particular the test chamber is attached enables a flexible and uncomplicated use of the apparatus, in particular a mobile use at different locations. The apparatus can be placed directly on a ground with the support surface. For this purpose, the rack can be designed to be levellable, i.e. in particular have at least one or more support feet that are arranged in a height-adjustable manner The rack is in particular of frame-like design and comprises in particular interconnected, in particular welded metal beams, in particular in the form of standardized profile elements. The rack is designed to be robust and cost-effective. In particular, all essential components, in particular the test chamber, are attached to the rack. The detachable fastening for repair and maintenance purposes is advantageous. It is advantageous to design the rack with corresponding receptacles for a forklift truck and/or lift truck. This simplifies the mobile transport of the apparatus. In addition or alternatively, lifting lugs can be fastened and/or are fastened laterally and/or on an upper side of the rack that is opposite the support surface in order to simplify the transport of the apparatus by means of a cargo crane, for example in a machine hall. It is also conceivable that a plurality of rollers are attached to the support surface in order to transport the apparatus with the rack directly on the ground, wherein at least one or more rollers can be designed to be rotationally drivable.

An evaluation unit comprising an evaluation unit for the, in particular automated, determination of operating parameters, wherein the evaluation unit comprises in particular a gas chromatograph enables the, in particular automated, verification of the operating parameters that are applied to a test quantity for degassing and/or deodorizing The evaluation unit serves to determine material properties and in particular the volatile components of the test quantity. In particular, the evaluation unit comprises a gas chromatograph to determine the proportion of volatile components in the bulk material.

A connection line directly connecting the pressurized gas line and the steam line, wherein in particular a regulating unit is arranged along the connection line, facilitates the combined addition of heated pressurized gas with water vapor into the test chamber. A regulating unit arranged along a connection line serves to adjust a ratio of heated pressurized gas, in particular heated pressurized air, and supplied moisture. The regulating unit can be used in particular to regulate the gas flow, in particular the air flow for degassing in the test chamber.

A method according to the invention has substantially the advantages of the apparatus. A test quantity that has been filled into the test chamber is degassed and/or deodorized by feeding a tempered and/or humidified pressurized gas into the test chamber. After a variably predeterminable period of time, a sample quantity is withdrawn from the test chamber via the withdrawal opening, in particular repeatedly and in particular at regular intervals. For this sample quantity, its material properties, in particular the volatile components, are determined. Based on changes in the material properties, the required operating parameters can be set and/or determined. If the predetermined material properties have been achieved, the operating parameters carried out with the apparatus can be transferred to the compounding and/or recycling facility. Otherwise, a new test series is started with modified operating parameters. The method is repeated in particular until the corresponding material properties of the bulk material have been achieved.

Both the features indicated in the patent claims and the features indicated in the following embodiment examples of the apparatus according to the invention are in each case suitable, alone or in combination with one another, for further refining the subject-matter according to the invention. The respective combinations of features do not represent any restriction with regard to the refinements of the subject-matter of the invention, but are essentially merely exemplary in character.

Further features, advantages and details of the invention will be apparent from the following description of embodiment examples based on the drawing.

### BRIEF DESCRIPTION OF THE DRAWING

FIG. 1 shows a schematic illustration of an apparatus according to the invention.

### DESCRIPTION OF THE PREFERRED EMBODIMENT

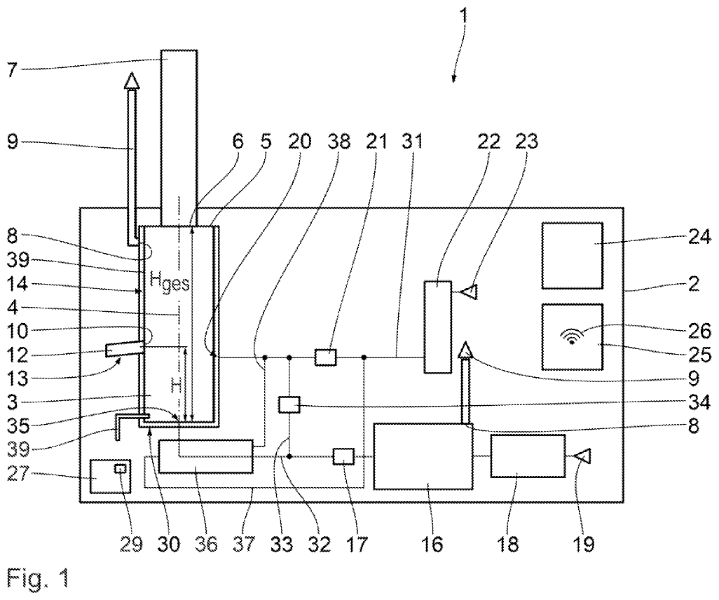

An apparatus marked 1 in FIG. 1 as a whole comprises a rack 2, which is shown purely schematically in FIG. 1. The rack 2 has a support surface with which the rack 2 and thus the apparatus 1 as a whole can be reliably and stably arranged on a ground. The rack 2 can be flat, in particular plate-like. The support surface is in particular square, in particular rectangular. The rack 2 can also be of frame-like design and have a plurality of interconnected, in particular welded, longitudinal beams. The longitudinal beams are in particular manufactured as standardized profile elements, in particular as I- or T-beams made of steel material.

The rack 2 can have height-adjustable support feet and/or castors on its support surface in a manner known per se. In addition, the rack 2 can have forklift receptacles and/or lifting lugs in order to simplify the transport of the rack 2. The apparatus 1 is suitable for mobile use.

A test chamber 3 is detachably secured to the apparatus 1. The test chamber 3 is substantially cylindrical in shape with a longitudinal axis 4 which is oriented in particular transversely and in particular perpendicularly to the support surface of the rack 2. In particular, the test chamber 3 is oriented vertically with the longitudinal axis 4.

The test chamber 3 has a housing 30 which is designed with double walls.

A vent opening 6 is arranged at the test chamber 3 in an upper region, in particular on the upper end face 5 of the test chamber 3. A vent line 7 is connected to the vent opening 6. With the vent line 7 the test chamber 3 can be connected to a central venting system. A filling opening for filling the plastic particles into the test chamber 3 can be formed in particular by the vent opening 6. It is possible, for example, to remove the vent line 7 from the vent opening 6 in order to add plastic granulate to the test chamber 3 through the vent opening 6.

Accordingly, the test chamber 3 can be emptied via the upper vent opening 6, for example by means of an extraction system. Alternatively, a separate emptying opening can be provided at the test chamber 3, in particular in the region of the lower end face, i.e. at the bottom of the test chamber 3.

In addition, a further vent opening 8 with a further vent line 9 is arranged at the double-walled housing 30. The vent opening 6 and the vent line 7 have a first line cross-section which is larger than a second line cross-section of the vent opening 8 and the vent line 9. The vent line 7 forms a main vent. The vent line 9 forms a secondary vent.

The test chamber 3 has a withdrawal opening 10, which is arranged in particular on the cylindrical jacket wall of the cylindrical test chamber 3. With respect to a lower end face 11 arranged opposite the upper end face 5, which forms the bottom of the test chamber 3, the withdrawal opening 10 is arranged in a height position H which corresponds approximately to half the total height H_{ges} of the test chamber 3. In particular, the following applies: H≤0.5*H_{ges}, in particular H≤0.4*H_{ges} and in particular H≤0.3*H_{ges}. The height positioning of the withdrawal opening 10 on the test chamber 3 ensures that bulk material can be reliably withdrawn from the test chamber, in particular even if the test chamber 3 is not completely filled with the test quantity.

A withdrawal unit is connected to the withdrawal opening 10, which, according to the embodiment example shown, comprises a withdrawal pipe 12 in the form of a, in particular cylindrical, downpipe. The withdrawal pipe 12 is in particular inclined with respect to the vertical with an angle of inclination of less than 30°. This ensures uncomplicated removal of bulk material from the test chamber 3, in particular by gravity. The withdrawal unit further has a shut-off element 13, which can be designed in particular as a shut-off slide and/or flap, in particular in the withdrawal pipe 12. With the shut-off element 13, the withdrawal pipe 12 can be shut off directly and a flow of bulk material therethrough can be prevented.

The test chamber 3 has a temperature control unit 14 which serves to control the temperature of an outer wall 39 of the test chamber 3. The temperature control unit 14 is in particular arranged directly at the test chamber 3 and is formed by a double-walled housing 30, i.e. by an outer pipe surrounding the test chamber 3. A temperature control fluid, in particular heated air, can be fed to this casing that surrounds the test chamber 3. A pressurized gas line 31 is connected to the double-walled housing 30 at a pressurized gas connection 20 in order to feed heated pressurized gas, in particular heated pressurized air.

The test chamber 3 has a feed opening 35 to which a steam generator 16 is connected by means of a steam line 32. The section of the steam line 32 that is connected to the feed opening 35 is also referred to as the gassing line. The feed opening 35 forms a steam connection to the test chamber 3. In addition, a steam volume regulator 17 is provided, which is arranged integrated in the steam generator 16 according to the embodiment example shown. Alternatively, it is possible that the steam volume regulator 17 is arranged separately and in particular downstream of the steam generator 16. The steam volume regulator 17 regulates the amount of steam supplied to the test chamber 3. A vent line 9 corresponding to the secondary vent 9 of the test chamber 3 is connected to the steam generator 16 at a vent opening 8.

An optional water softening unit 18 is connected upstream of the steam generator 16. The water softening unit 18 enables the use of drinking water, in particular water containing lime, for steam generation. A water source 19 can be connected upstream of the water softening unit 18. The water source 19 can be implemented by a water connection to a water pipe network. The use of the apparatus 1 is thereby simplified. In particular, elaborate provisions for a water source can be dispensed with.

In addition or alternatively, a water tank can be provided as a water source for a corresponding water pipe connection. By means of a water tank, the apparatus is suitable for self-sufficient use. In particular, a water pipe network for the water supply is dispensable.

A gas flow regulator 21, in particular an air flow regulator, is connected to the pressurized gas line 31. A heating element 22, in particular an air heater, is arranged upstream of the gas flow regulator 21. A pressurized gas source 23 can be connected to the heating element 22. The pressurized gas source 23 can be a pressurized air line network, which is typically available in a machine hall anyway. Additionally or alternatively, the pressurized gas source 23 may be designed as a compressor. With the compressor, the apparatus is suitable for mobile, in particular self-sufficient, use.

The pressurized gas line 31 is connected to the steam line 32 by means of a connection line 33. A regulating unit 34 is arranged along the connection line 33. By means of the connection line 33 it is possible to feed, in particular heated, pressurized gas from the pressurized gas line 31 via the connection line 33 to the steam line 32 and to feed it via the gassing line and the feed opening 35 to the test chamber 3. In this case, the feed opening 35 is a gassing connection.

The regulating unit 34 can be used to adjust the proportion of heated pressurized gas that is fed to the steam line 32. With the regulators 17, 21 and 34 it is thus possible to add either only heated pressurized gas or only water vapor or both in combination via the feed opening 35 into the test chamber 3.

The apparatus 1 has a central voltage supply 24, which is in particular permanently connected to the apparatus 1. The central voltage supply 24 provides the electricity that is required for the operation of the apparatus.

The apparatus 1 has a central control unit 25 which is in signal connection, in particular bidirectional signal connection, in particular with the steam generator 16, the steam volume regulator 17, the gas flow regulator 21, the gas heater 22 and the temperature control unit 14 by means of a signal connection 26 which is shown purely symbolically in FIG. 1. The control unit 25 enables the uncomplicated and immediately variable definition of the operating parameters for the degassing and deodorization of the test quantity in the test chamber 3.

The apparatus 1 comprises an evaluation unit 27 which includes a gas chromatograph 29. The evaluation unit 27 is suitable for determining the operating parameters for degassing in an automated manner.

The evaluation unit 27 can be permanently connected to the apparatus 1. Alternatively, the evaluation unit 27 can be designed mechanically separate from the apparatus 1, in particular from the rack 2.

An insulating element 36 is arranged along the steam line 32 downstream of the steam volume regulator 17. The insulating element 36 is designed to be of a hollow shape, in particular of a substantially cuboid shape. The insulating element 36 is of box-like design. The insulating element 36 is also referred to as an insulating box.

The gassing line is passed through the insulating box 36, wherein a partial flow is led via a branch line 37 in cross flow to the gassing line through the insulating box 36. The branch line 37 is a bypass line and serves to reduce heat losses of the gas flow into the test chamber 3. The gas that is fed into the insulating box 36 via the branch line 37 is discharged from the insulating box 36 via the return line 38 and fed to the pressurized gas line 31.

Another bypass line not shown, which has a larger line cross-section compared to the bypass line 37, can be used to preheat the sample chamber 3 with heated air and/or water vapor. Alternatively, a separate heater, in particular in the form of a heating blanket, may be attached to the outer wall 39 and used to preheat the test chamber 3.

A drainage line 39 is connected to the housing 30. Via the drainage line 39, condensate that has formed in the double-walled housing 30 can be drained off in a targeted manner and, in particular, be drained off automatically due to the force of gravity. The drainage line 39 can be designed to be closable with a blocking element.

A method for determining the operating parameters for degassing and/or deodorizing a test quantity is explained in more detail below.

In the apparatus 1 provided, a test quantity of a defined amount, for example 10 l, is filled into the test chamber 3. Subsequently, the test quantity in the test chamber is degassed and/or deodorized at defined operating parameters, i.e. in particular at a first pressure provided by the gas flow regulator 21, at a first temperature provided by the heating element 22, at a defined gas quantity provided by the gas flow regulator 21, at a defined humidity provided by the steam flow regulator 17 and/or over a defined degassing period, which is also specified in particular by the central control unit 25. In particular, the test quantity in the test chamber 3 is degassed and/or deodorized using heated gas and/or with the addition of steam.

After completion of the degassing and/or deodorization cycle, bulk material, in particular a partial quantity of the test quantity, is withdrawn from the test chamber 3 by means of the withdrawal pipe 12 and analyzed by means of the evaluation unit 27. The evaluation unit 27 is used to examine predeterminable material properties. For this purpose, the evaluation unit has in particular an odour sensor 28 and a gas chromatograph 29.

Depending on the material properties determined, a new degassing and/or deodorization cycle can be carried out or the previously applied operating parameters can be qualified as suitable.

The method is uncomplicated, simple and not very cost-intensive.

1. An apparatus for determining operating parameters for at least one of degassing and deodorizing plastic particles, wherein the apparatus comprises at least one test chamber for at least one of degassing and deodorizing a test quantity of plastic particles, wherein each test chamber has
a. a filling opening for filling in the test quantity,
b. a withdrawal opening for withdrawing a sample quantity of the plastic particles,
c. a vent opening for venting the test chamber,
wherein connected to a feed opening of the test chamber are at least one of
d. a pressurized gas line for feeding pressurized gas and a heating element and,
e. a steam line for feeding steam and a steam generator.

2. The apparatus according to claim 1, comprising a gas flow regulator that is connected to the pressurized gas line.

3. The apparatus according to claim 1, comprising a pressurized gas source that is connectable to the pressurized gas line.

4. The apparatus according to claim 1, wherein the steam generator has a vent opening.

5. The apparatus according to claim 1, comprising a water softening unit that is connected to the steam generator.

6. The apparatus according to claim 1, comprising a steam volume regulator that is connected to the steam line.

7. The apparatus according to claim 1, comprising a water source that is connectable to the steam generator.

8. The apparatus according to claim 1, comprising a vent line that is connectable to the vent opening for connecting the test chamber to a central venting system.

9. The apparatus according to claim 1, comprising a withdrawal unit connected to the one withdrawal opening.

10. The apparatus according to claim 9, wherein the withdrawal unit comprises a withdrawal pipe, which can be shut off by means of a shut-off element.

11. The apparatus according to claim 10, wherein the withdrawal pipe is a downpipe.

12. The apparatus according to claim 1, comprising a control unit which is designed to control operating parameters.

13. The apparatus according to claim 1, comprising a temperature control unit for controlling the temperature of an outer wall of the test chamber, wherein the temperature control unit comprises a double-walled housing surrounding one of the test chamber and a separate heater.

14. The apparatus according to claim 1, comprising a rack supporting the apparatus, to which rack the test chamber is attached.

15. The apparatus according to claim 1, comprising an evaluation unit for the determination of operating parameters.

16. The apparatus according to claim 15, wherein the evaluation unit comprises a gas chromatograph.

17. The apparatus according to claim 1, wherein the pressurized gas line and the steam line are directly connected by means of a connection line.

18. The apparatus according to claim 17, wherein a regulating unit is arranged along the connection line.

19. A method for determining operating parameters for at least one of degassing and deodorizing plastic particles comprising the method steps of
providing an apparatus for determining operating for at least one of degassing and deodorizing plastic particles, wherein the apparatus comprises at least one test chamber for at least one of degassing and deodorizing a test quantity of plastic particles, wherein each test chamber has
a. a filling opening for filling in the test quantity,
b. a withdrawal opening for withdrawing a sample quantity of the plastic particles,
c. a vent opening for venting the test chamber, wherein connected to a feed opening of the test chamber are at least one of
d. a pressurized gas line for feeding pressurized gas and a heating element and
e. a steam line for feeding steam and a steam generator,
filling a test quantity of plastic particles into the test chamber,
at least one of degassing and deodorizing the test quantity in the test chamber by feeding at least one of temperature-controlled pressurized gas and steam via a feed opening of the test chamber,
removing a sample quantity of at least one of the degassed and deodorized plastic particles from the test chamber,
determining material properties of the sample quantity,
determining operating parameters for at least one of degassing and deodorizing on the basis of the determined material properties.
